(19) United States
(12) Patent Application Publication
(10) Pub. No.: US 2025/0312617 A1
(43) Pub. Date: Oct. 9, 2025

(54) Therapy Kit

(71) Applicant: SHENZHEN KAIYAN MEDICAL EQUIPMENT CO., LTD, Shenzhen (CN)

(72) Inventors: ALAIN DIJKSTRA, Amstelveen (NL); LI XIANG, Shenzhen (CN); Wen Caiyun, Shenzhen (CN)

(73) Assignee: SHENZHEN KAIYAN MEDICAL EQUIPMENT CO., LTD, Shenzhen (CN)

(21) Appl. No.: 18/641,512

(22) Filed: Apr. 22, 2024

(30) Foreign Application Priority Data
Apr. 8, 2024 (CN) 2024207136406

(51) Int. Cl. A61N 5/067; A61N 1/04; A61N 1/36; A61N 5/06

(52) U.S. Cl. CPC A61N 5/067; A61N 1/0452; A61N 1/0456; A61N 1/0492; A61N 1/36021; A61N 1/36034; A61N 2005/0626; A61N 2005/0634; A61N 2005/0645; A61N 2005/0651; A61N 2005/066; A61N 2005/0661; A61N 2005/0663

(57) ABSTRACT

Embodiments of the present invention provide a therapy kit. The therapy kit includes one or more therapeutic patches including one or more stimulation elements. Furthermore, the therapy kit includes one or more controllers corresponding to the one or more respective therapeutic patches, the one or more controller configured to control activation, deactivation, and operational characteristics of the one or more stimulation elements. The therapy kit also includes a housing container. The housing container further includes a base portion including an electrical power source, one or more controller cavities configured to receive the one or more respective controllers, and one or more patch cavities configured to receive the one or more respective therapeutic patches. Furthermore, the one or more controllers are configured to receive electrical power from the electrical power source. Also, the one or more therapeutic patches are configured to receive electrical power from the one or more respective controllers.

### TECHNICAL FIELD

The present invention relates generally to therapeutic and recreational devices. More specifically, the present invention relates to devices that can deliver multiple kinds of stimulation such as electrical, heating, cooling, and irradiation, can be conveniently stored and transported, and utilized whenever required both in indoor conditions and outdoor conditions.

### BACKGROUND ART

Therapeutic devices of several kinds for therapeutic and recreational purposes have been known in the art for quite some time. Such devices use electrodes, heating elements, cooling elements, and irradiation sources such as Light Emitting Diodes (LEDs) and lasers to deliver energy in one form or another to a portion of the body for pain relief, muscle relaxation, skin rejuvenation, neural stimulation, etc. However, such therapeutic devices are generally bulky in construction and require large amounts of electrical power. Therefore, to meet the demands of electrical power they are generally equipped with relatively large rechargeable batteries or are AC-powered with rectifiers, op-amps, transformers, and filters built in to convert AC power into DC power. As a consequence, the weight and size of such devices make them difficult to carry around and be used in outdoor conditions.

Therefore, there is a need in the art, for a therapy kit that overcomes the disadvantages and limitations associated with the prior art and provides a more satisfactory solution.

### OBJECTS OF THE INVENTION

Some of the objects of the invention are as follows:

An object of the present invention is to provide a therapy kit that includes portable therapeutic patches and controllers corresponding to the therapeutic patches in a housing container that can be conveniently transported and utilized in both indoor and outdoor conditions.

Another object of the present invention is to provide a therapy kit in which the controllers are configured to store electrical power and can be charged while being located in the housing container.

Another object of the present invention is to provide a therapy kit in which the housing container includes a rechargeable electrical power source that can be charged through wired or wireless means.

Another object of the present invention is to provide a therapy kit in which the therapeutic patches can be used for multiple purposes such as electrical stimulation, heating, cooling, vibratory massage, etc.

Another object of the present invention is to provide a therapy kit in which the therapeutic patches are configured to provide localized therapy to several different portions of the body of a user.

Another object of the present invention is to provide a therapy kit that can communicate with a user computing device to receive control input for modifying the operational characteristics of the therapeutic patches.

Also, it is an object of the present invention that the control input may also be received through a user interface provided with the housing container.

### SUMMARY OF THE INVENTION

According to a first aspect of the present invention, there is provided a therapy kit. The therapy kit includes one or more therapeutic patches including one or more stimulation elements. Furthermore, the therapy kit includes one or more controllers corresponding to the one or more respective therapeutic patches, the one or more controller configured to control activation, deactivation, and operational characteristics of the one or more stimulation elements. The therapy kit further includes a housing container. The housing container includes a base portion including an electrical power source. The base portion defines therewithin one or more controller cavities configured to receive the one or more respective controllers, and one or more patch cavities located above the respective one or more controller cavities, the one or more patch cavities configured to receive the one or more respective therapeutic patches. The housing container also includes a top lid configured to be located above the base portion. Furthermore, the one or more controllers are configured to receive electrical power from the electrical power source through base power terminals provided within each one of the one or more respective controller cavities. Also, the one or more therapeutic patches are configured to receive electrical power from the one or more respective controllers, through patch power terminals provided with each one of the one or more respective therapeutic patches.

In one embodiment of the invention, the therapy kit further includes one or more stickers, each one of the one or more stickers provided with adhesive agents on two respective opposing surfaces for attaching the one or more respective therapeutic patches to the skin of a user.

In one embodiment of the invention, the adhesive agents provided on the two respective opposing surfaces of at least one of the one or more stickers are diaphanous.

In one embodiment of the invention, the therapy kit further includes an intermediate lid configured to pivot between an open position and a closed position, such that, when in the closed position, the intermediate lid is located above the one or more patch cavities. Furthermore, the intermediate lid includes one or more sticker cavities configured to receive the one or more respective stickers.

In one embodiment of the invention, the intermediate lid includes an intermediate lid top portion and an intermediate lid bottom portion. The intermediate lid top portion includes the one or more sticker cavities.

In one embodiment of the invention, the intermediate lid bottom portion includes a mirror fastened to the intermediate lid.

In one embodiment of the invention, the intermediate layer is pivotably coupled to the base portion and/or the top lid.

In one embodiment of the invention, the intermediate lid includes one or more lid coupling units configured to couple the intermediate lid with the top lid when the intermediate lid is in the open position.

In one embodiment of the invention, each one of the one or more therapeutic patches comprises an adhesive agent on a surface configured to be attached to skin of the user.

In one embodiment of the invention, the adhesive agent of the one or more therapeutic patches is diaphanous.

In one embodiment of the invention, the therapy kit further includes controller magnetic elements in each one of the one or more controllers and controller cavity magnetic elements in under the one or more respective controller cavities, to enable magnetic couplings between the one or more controllers and the one or more respective controller cavities.

In one embodiment of the invention, each one of the one or more controllers includes one or more respective rechargeable controller batteries and one or more respective second State of Charge (SoC) indicators, the one or more second SoC indicators configured to indicate SoC of the one or more respective rechargeable controller batteries.

In one embodiment of the invention, the electrical power source of the housing container includes one or more rechargeable batteries, and the housing container includes a first State of Charge (SoC) indicator configured to indicate an SoC of the one or more rechargeable batteries.

In one embodiment of the invention, each one of the one or more controller cavities includes a controller ejection enabling feature to enable removal of the one or more controllers from the one or more respective controller cavities.

In one embodiment of the invention, each one of the one or more controller cavities includes a mating feature configured to mate with a complementary mating feature of a respective controller of the one or more controllers, to prevent relative rotation between the one or more controller cavities and the one or more respective controllers.

In one embodiment of the invention, the electrical power source of the base portion container includes one or more rechargeable batteries, the base portion including a receiving inductor coil configured to receive power from a time-varying magnetic field generated by a transmitter inductor coil of a wireless charging device.

In one embodiment of the invention, the one or more stimulation elements are selected from a group consisting of irradiation sources, heating elements, cooling elements, vibration elements, ultrasonic wave generators, electrodes, and combinations thereof.

In one embodiment of the invention, the irradiation sources are configured to emit electromagnetic radiation in a wavelength range of 300 nm to 1200 nm.

In one embodiment of the invention, the top lid is pivotably coupled to the base portion.

In one embodiment of the invention, the housing container further includes a charging port configured to receive electrical power from an external power source.

In one embodiment of the invention, the one or more controllers further include one or more respective communication interfaces configured to receive control input signals from a user computing device, through a communication network, to modify operational characteristics of the one or more stimulation elements.

In one embodiment of the invention, the one or more controllers further include one or more respective user interfaces configured to receive control input signals to modify the operational characteristics of the one or more stimulation elements.

According to a second aspect of the present invention, there is provided a therapy kit. The therapy kit includes one or more therapeutic patches including one or more stimulation elements. The therapy kit further includes one or more stickers, each one of the one or more stickers provided with adhesive agents on two respective opposing surfaces for attaching the one or more respective therapeutic patches to the skin of a user. Furthermore, the therapy kit includes one or more controllers corresponding to the one or more respective therapeutic patches, the one or more controllers configured to control activation, deactivation, and operational characteristics of the one or more stimulation elements. The therapy kit also includes a housing container. The housing container includes a base portion including an electrical power source. The base portion defines therewithin one or more controller cavities configured to receive the one or more respective controllers, and one or more patch cavities located above the respective one or more controller cavities, the one or more patch cavities configured to receive the one or more respective therapeutic patches. The housing container further includes an intermediate lid configured to pivot between an open position and a closed position, such that, when in the closed position, the intermediate lid is located above the one or more patch cavities, wherein the intermediate lid includes one or more sticker cavities configured to receive the one or more respective stickers. The housing container also includes a top lid configured to be located above the base portion and the intermediate lid. The one or more controllers are configured to receive electrical power from the electrical power source through base power terminals provided within each one of the one or more respective controller cavities, Furthermore, the one or more therapeutic patches are configured to receive electrical power from the one or more respective controllers, through patch power terminals provided with each one of the one or more respective therapeutic patches. The intermediate lid includes an intermediate lid top portion and an intermediate lid bottom portion, and the intermediate lid top portion includes the one or more sticker cavities. The intermediate lid bottom portion includes a mirror fastened to the intermediate lid. Also, the one or more controllers further include one or more respective user interfaces configured to receive control input signals to modify the operational characteristics of the one or more stimulation elements.

In one embodiment of the invention, the one or more controllers further include one or more respective communication interfaces configured to receive control input signals from a user computing device, through a communication network, to modify operational characteristics of the one or more stimulation elements.

According to a third aspect of the present invention, there is provided a method of utilizing a therapy kit. The method includes locating one or more controllers in one or more respective controller cavities of a housing container of the therapy kit, such that, the one or more controllers receive electrical power from an electrical power source, through base power terminals provided within each one of the one or more respective controller cavities. The method further includes removing one or more therapeutic patches from one or more respective patch cavities from one or more respective patch cavities and attaching the one or more therapeutic patches onto the skin of a user. Furthermore, the method includes attaching the one or more controllers to the one or more respective therapeutic patches including one or more stimulation elements, such that, the one or more therapeutic patches receive electrical power through patch power terminals provided with each one of the one or more respective therapeutic patches. Also, the method includes controlling activation, deactivation, and operational characteristics of the one or more stimulation elements using the one or more respective controllers.

In one embodiment of the invention, the one or more therapeutic patches are attached to the skin of the user through one or more respective stickers, each one of the one or more stickers provided with adhesive agents on respective opposite surfaces of the one or more stickers.

In one embodiment of the invention, each one of the one or more therapeutic patches includes an adhesive agent on a surface configured to be attached to the skin of the user.

In one embodiment of the invention, the method further includes receiving, by the one or more controllers, control input signals through one or more respective user interfaces to modify operational characteristics of the one or more stimulation elements.

In one embodiment of the invention, the method further includes receiving, by the one or more controllers, control input signals from a user computing device, through one or more respective communication interfaces and a communication network, to modify operational characteristics of the one or more stimulation elements.

In one embodiment of the invention, the method further includes pivoting an intermediate lid of the housing container, from a closed position to an open position to reveal a mirror fastened to an intermediate lid bottom portion of the intermediate lid.

In the context of the specification, the term “processor” refers to one or more of a microprocessor, a microcontroller, a general-purpose processor, a Field Programmable Gate Array (FPGA), a Neural Processing Unit (NPU), a Graphics Processing Unit (GPU), a Tensor Processing Unit (TPU), an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying between the Ultraviolet (UV) frequencies and Infrared (IR) frequencies and wavelengths (including all visible light frequencies and wavelengths), wherein the range is inclusive of UV and IR frequencies and wavelengths. It is to be noted here that UV radiation can be categorized in several manners depending on respective wavelength ranges, all of which are envisaged to be under the scope of this invention. For example, UV radiation can be categorized as, Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm) Similarly, IR radiation may also be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, the term “polymer” or “plastic” refers to a material made up of long chains of organic molecules (having eight or more organic molecules) including, but not limited to, carbon, nitrogen, oxygen, and hydrogen as their constituent elements. The term polymer is envisaged to include both naturally occurring polymers such as wool, and synthetic polymers such as polyethylene and nylon.

In the context of the specification, the phrase “diaphanous material” refers to a material that allows at least a portion of one or more forms of electromagnetic radiation (such as Infrared, Ultraviolet, X-rays, Visible Light, Microwaves, Radio Waves, etc.) to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

In the context of the specification, “Light Emitting Diodes (LEDs)” refer to semiconductor diodes capable of emitting electromagnetic radiation when supplied with an electric current. LEDs are characterized by their superior power efficiencies, smaller sizes, rapidity in switching, physical robustness, and longevity when compared with incandescent or fluorescent lamps. In that regard, one or more LEDs may be through-hole type LEDs (generally used to produce electromagnetic radiations of red, green, yellow, blue, and white colors), Surface Mount Technology (SMT) LEDs, Bi-color LEDs, Pulse Width Modulated RGB (Red-Green-Blue) LEDs, and high-power LEDs, etc.

Materials used in the LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium-doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a top-perspective view of a therapy kit, in accordance with an embodiment of the present invention;

FIG. 1B illustrates a back perspective view of the therapy kit of FIG. 1A;

FIG. 1C illustrates an exploded view of the therapy kit in FIG. 1A, when viewed from the top;

FIG. 1D illustrates an exploded view of the therapy kit in FIG. 1A, when viewed from the bottom;

FIG. 1E illustrates a housing container of the therapy kit of FIG. 1A, in a first partially open configuration;

FIG. 1F illustrates the housing container of FIG. 1E in a second partially open configuration;

FIG. 2A illustrates an exploded view of a base portion of the housing container of FIG. 1E;

FIG. 2B illustrates an exploded view of a controller of the therapy kit of FIG. 1A;

FIG. 3A illustrates an exploded view of a therapy kit when viewed from the top, in accordance with another embodiment of the present invention;

FIG. 3B illustrates a partially exploded view of the therapy kit in FIG. 3A, when viewed from the front;

FIG. 4 illustrates a method of utilizing the therapy kit, in accordance with an embodiment of the present invention;

FIG. 5 illustrates an example application scenario of the therapy kit of FIGS. 1A-2B; and

FIG. 6 illustrates an example application scenario of the therapy kit of FIGS. 3A and 3B.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention provide a therapy kit. The therapy kit includes one or more therapeutic patches and one or more respective controllers to control the activation, deactivation, and operational characteristics of the therapeutic patches. The therapeutic patches and the controllers are housed in a single housing container including a top lid and a base portion. In that regard, the base portion includes cavities to locate the controllers (controller cavities) and the therapeutic patches (patch cavities). The base portion also includes an electrical power source with rechargeable batteries that are used to recharge rechargeable batteries of the controllers through power terminals provided in the cavities designed for the controllers. Furthermore, magnets (permanent magnets or electromagnets) may be provided under controller cavities and within the controllers to enable mechanical magnetic couplings between the controllers and the respective controller cavities. Mating features may be provided in the controller cavities and complementary mating features may be provided on the controllers to prevent relative rotation between the controllers and the respective controller cavities. Furthermore, the design of the controller cavities may be modified, for example by providing extended tapered sections to enable convenient ejection of the controllers from the controller cavities.

The therapeutic patches are located in the patch cavities above the controller cavities. The therapeutic patches are provided with several stimulation elements such as irradiation sources, heating elements, cooling elements, vibratory elements, ultrasonic wave generators, electrodes, and the like. Furthermore, the therapeutic patches are configured to be attached to the skin of a user either directly or through stickers. The stickers may be located in sticker cavities provided in the upper portion of an intermediate lid located between the top lid and the base portion. A lower portion of the intermediate lid may be provided with a mirror to aid in the location of the therapeutic patches on the skin of the user. Once the therapeutic patches have been attached to the skin of the user, the controllers are attached to each one of the therapeutic patches. The therapeutic patches receive electrical power from the rechargeable batteries of the controllers through patch power terminals. Furthermore, the operational characteristics of the stimulation elements may be controlled using user interfaces provided on the controllers or wirelessly using a computing device such as a smartphone, a tablet PC, a notebook PC, a desktop PC, and the like.

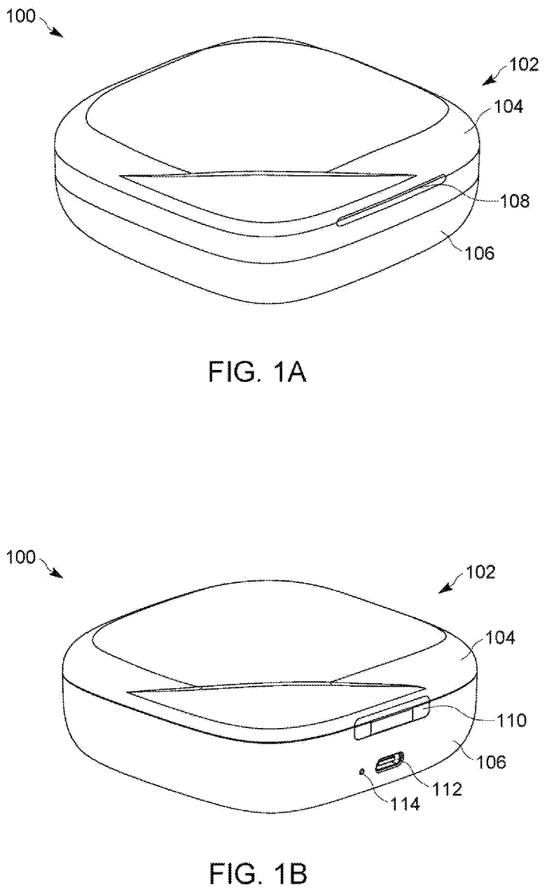

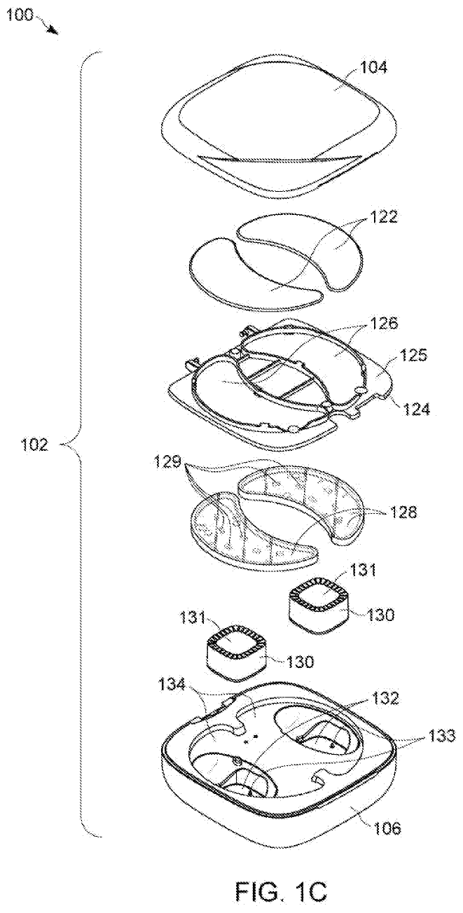

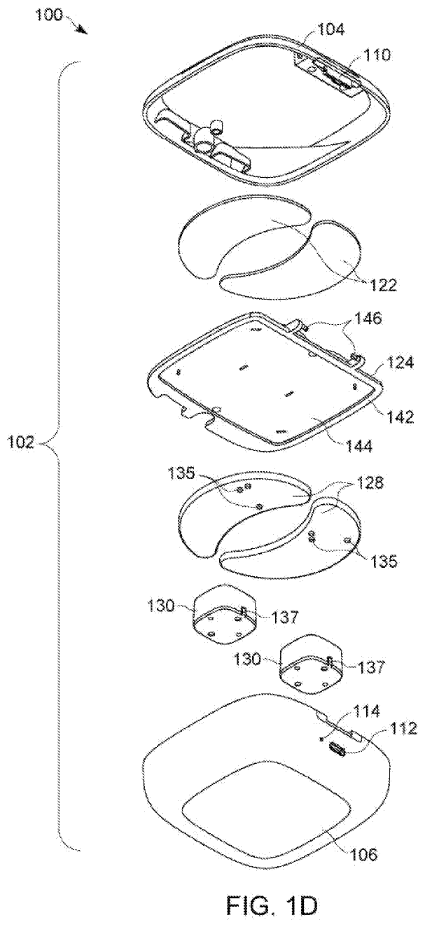

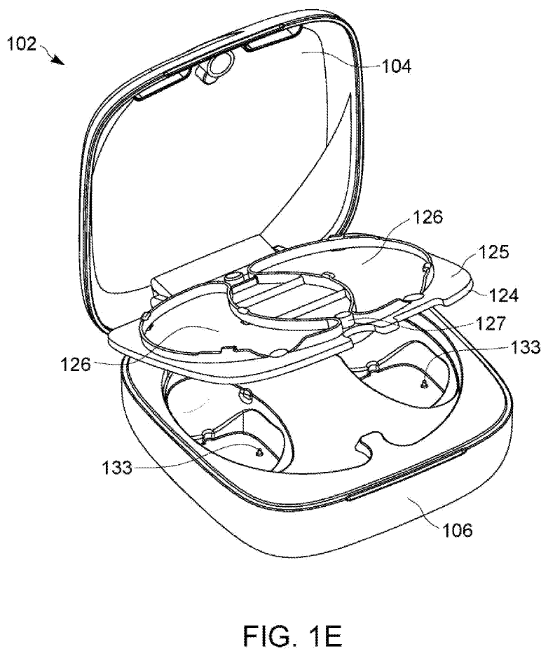

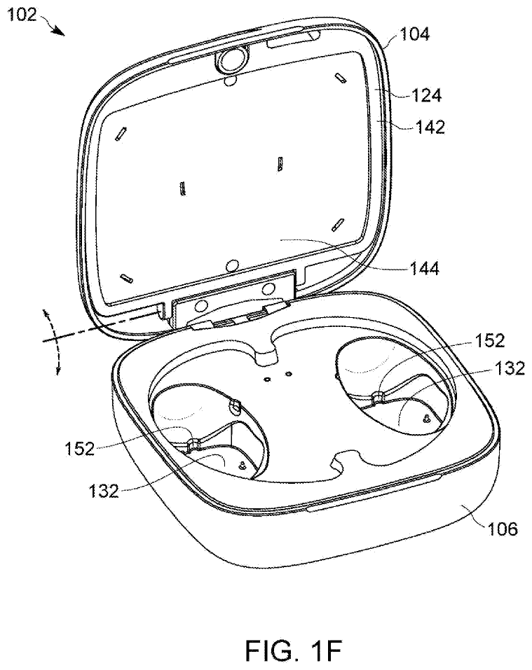

Several embodiments of the present invention will now be elucidated in detail with the help of FIGS. 1A-6 as references.

FIG. 1A illustrates a top perspective view of a therapy kit 100, in accordance with an embodiment of the present invention. FIG. 1B illustrates a back perspective view of the therapy kit 100 of FIG. 1A. The therapy kit 100 includes a housing container 102. The housing container 102 may at least in part be made from a rigid and durable polymer material. Some of the materials that the housing container 102 may be made of include Polycarbonate (PC), Polymethyl methacrylate (PMMA), Polyvinyl chloride (PVC), Acrylonitrile Butadiene Styrene (ABS), High-density polyethylene (HDPE), Nylon or Polyamide (PA), Polytetrafluoroethylene (PTFE), Polypropylene (PP), and the like. The hosing container 102 includes a top lid 104 and a base portion 106. The top lid 104 is configured to be located above the base portion 106. A slot 108 has been provided on the front side of the housing container 102, between the top lid 104 and the base portion 106 to enable a user to conveniently detach the top lid 104 from the base portion 106.

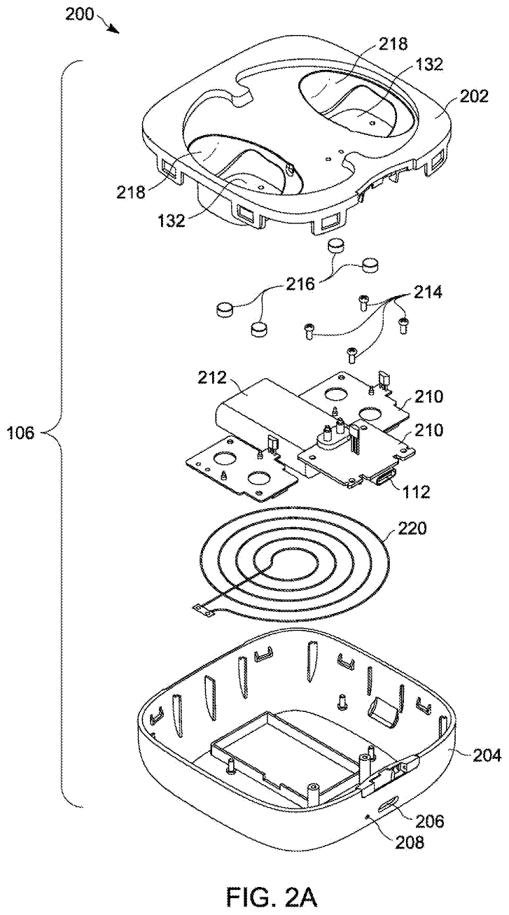

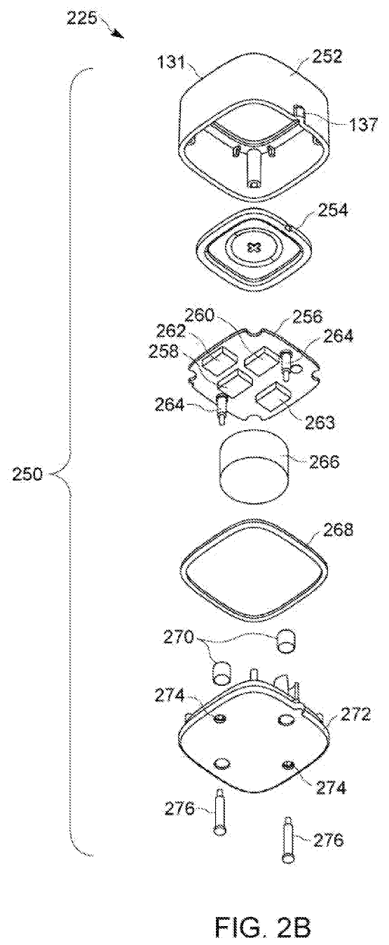

Furthermore, the top lid 104 is pivotably coupled with the base portion 106 on the rear side of the housing container 102, through a hinge joint 110. In that regard, the top lid 104 is configured to pivot between an open position and a closed position. In FIGS. 1A and 1B, the top lid 104 is in the closed position. Also provided with the base portion 106 is a charging port 112 configured to receive electrical power for an electrical power source (See FIG. 2A) located within the base portion 106, from an external power source (not shown). The charging port 112, in that regard, may be selected from a group consisting of USB-A, B, C type ports, or any other compatible charging port. Furthermore, the base portion 106 includes a first State of Charge (SoC) indicator 114 configured to indicate an SoC of the electrical power source (See FIG. 2A) of the base portion 106.

FIG. 1C illustrates an exploded view of the therapy kit 100 of FIG. 1A, when viewed from the top. FIG. 1D illustrates an exploded view of the therapy kit 100 of FIG. 1A, when viewed from the bottom. Furthermore, the base portion 106 defines therewithin one or more patch cavities 134. The one or more patch cavities 134 are configured to receive therewithin one or more therapeutic patches 128. The one or more therapeutic patches 128 include one or more stimulation elements 129. In several embodiments of the invention, the one or more stimulation elements 129 may be selected from a group consisting of irradiation sources, heating elements, cooling elements, vibration elements, ultrasonic wave generators, electrodes, and combinations thereof.

Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation. Furthermore, it is envisaged that the one or more therapeutic patches 128 would be made at least in part from a diaphanous material. For example, the one or more therapeutic patches 128 may include an adhesive agent for attaching the one or more therapeutic patches 128 to the skin of the user. In that regard, it is envisaged that at least portions of the one or more therapeutic patches 128 facing the skin and the adhesive agent would be diaphanous. It is further envisaged that the adhesive agent of the one or therapeutic patches 128 be skin-friendly. Some of the examples of skin-friendly adhesive agents may include but are not limited to, silicones polydimethylsiloxane (PDMS) and silicone acrylates, acrylics, hydrocolloids, cyanoacrylates, etc.

In the case of a stimulation element being a vibration element, the stimulation element would be a vibrating head connected to an eccentric mass rotating motor or a linear resonant motor. In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the body of a user. TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Furthermore, the base portion 106 includes one or more controller cavities 132. The one or more controller cavities 132 are located below the one or more patch cavities 134. The one or more controller cavities 132 are configured to receive therewithin one or more controllers 130. The one or more controllers 130 may include one or more respective rechargeable controller batteries (See FIG. 2B) configured to power the one or more stimulation elements 129 of the one or more therapeutic patches 128. In that regard, each one of the one or more controller cavities 132 includes base power terminals 133. The one or more controllers 130 are configured to receive electrical power from the electrical power source (See FIG. 2A) of the base portion 106 through the base power terminals 133 to charge the one or more respective rechargeable controller batteries. The one or more controllers 130 may further include processors, memory units, and communication interfaces (See FIG. 2B) to modify, control, and monitor operational characteristics of the one or more stimulation elements 129 by controlling the amount of electrical power being delivered to the one or more stimulation elements 129. For instance, the one or more controllers 130 may include one or more respective user interfaces 131 configured to receive control input signals to modify operational characteristics of the one or more stimulation elements. In that regard, the one or more user interfaces 131 may be embodied as a touch interface (resistive or capacitive type), a single control button, or a group of control toggles.

In several embodiments of the invention, the base power terminals 133 may be embodied as spring-loaded dowel-pins. Also, the one or more therapeutic patches 128 are configured to receive electrical power from the one or more respective controllers 130 through patch power terminals 135 provided with each one of the one or more therapeutic patches 128. In several embodiments of the invention, the patch power terminals 135 may also be embodied as spring-loaded dowel pins. The one or more controllers 130 also include one or more respective controller protrusions that act as complementary mating features 137 for mating with mating features (See FIG. 1F) of the one or more respective controller cavities 132 to prevent relative rotation between the one or more controller cavities 132 and the one or more respective controllers 130.

The therapy kit 100 further includes an intermediate lid 124. The intermediate lid 124 is also configured to pivot between an open position and a closed position. When in the closed position, the intermediate lid 124 is configured to be located above the one or more patch cavities 134. In FIG. 1C, the intermediate lid 124 is in a partially open position. Furthermore, an intermediate lid top portion 125 is exposed. The intermediate lid top portion 125 includes one or more sticker cavities 126. The one or more sticker cavities 126 are configured to receive therewithin one or more respective stickers 122. Each one of the one or more stickers 122 includes adhesive agents provided on both opposing faces of each one of the one or more stickers 122. The one or more stickers 122 are configured to attach one or more respective therapeutic patches 128 to the skin of the user in embodiments where the one or more therapeutic patches 128 may not be provided with adhesive agents themselves.

In several embodiments of the invention, the adhesive agents on both the opposing faces of the one or more stickers 122 may be identical and be selected from skin-friendly adhesive agents cited in the aforementioned discussion. Alternately, the adhesive agents only on one face configured to contact the skin of the user may need to be skin-friendly. In embodiments where the one or more stimulation elements 129 include irradiation sources, materials of the one or more stickers 122, and the adhesive agents may also be diaphanous. In FIG. 1C, an intermediate lid bottom portion 142 of the intermediate lid 124 is exposed. The intermediate lid bottom portion 142 includes a mirror 144 fastened to the intermediate lid 124. The fastening of the mirror 144 with the intermediate lid 124 may be achieved with the help of adhesives, threaded fasteners, snap-fit fastening arrangements, rivets, double-sided tapes, and the like. Intermediate lid protrusions 146 on the rear side of the intermediate lid 124 allow the intermediate lid 124 to be pivotably coupled to the base portion 106 and/or the top lid 104 using the same hinge joint 110 or a distinct hinge joint.

FIG. 1E illustrates the housing container 102 of the therapy kit 100 of FIG. 1A, in a first partially open configuration. FIG. 1F illustrates the housing container 102 of FIG. 1E in a second partially open configuration. The intermediate lid 124 further includes one or more lid coupling units 127 configured to couple the intermediate lid 124 with the top lid 104 when the intermediate lid 124 is in the open position as illustrated in FIG. 1F. The one or more lid coupling units 127 may be enabled by magnets, adhesives, snap-fit arrangements, clips, or any other equivalent non-permanent coupling arrangements. Also illustrated in FIG. 1F are indentations that act as mating features 152 of the one or more controller cavities 132. The mating features 152 are configured to mate with the complementary mating features 137 of the one or more controllers 130 to prevent relative rotation between the one or more controller cavities 132 and the one or more respective controllers 130.

FIG. 2A illustrates an exploded view 200 of the base portion 106 of the housing container 102 of FIG. 1E. The base portion 106 includes an upper shell cap 202 that is configured to mate with a lower shell cap 204 of the base portion 106. The mating may be achieved using threaded fasteners, rivets, snap-fit arrangements, clips, and the like. The upper shell cap 202 defines therewithin the one or more controller cavities 132. Furthermore, one or more extended tapered indentations have been provided surrounding the one or more respective controller cavities 132. The one or more extended tapered indentations act as controller ejection enabling features 218. The controller ejection enabling features 218 allow a user to insert their fingers up to the one or more controller cavities 132 and under top surfaces of the one or more respective controllers 130, thereby enabling removal of the one or more controllers 130 from the one or more respective controller cavities 132. Furthermore, under each one of the one or more controller cavities 132 are located controller cavity magnetic elements 216 that couple with controller magnetic elements (See FIG. 2B) of the one or more controllers 130 to allow magnetic couplings between the one or more controllers 130 and the one or more respective controller cavities 132.

Furthermore, in between the upper shell cap 202 and the lower shell cap 204, there is located an electrical power source 212 mounted to base Printed Circuit Boards (PCBs) 210. The electrical power source 212 may include one or more rechargeable batteries configured to be charged through the charging port 112. The first SoC indicator 114 in that regard, may be configured to indicate the SoC of the one or more rechargeable batteries of the electrical power source 212. The base PCBs 210 may be fastened to the lower shell cap 204 using fasteners 214 which may be threaded fasteners such as screws or bolts. The lower shell cap 204 further includes a first aperture 206 for accommodating the charging port 112 and a second aperture 208 for accommodating the first SOC indicator 114. The base portion 106 also includes a receiving inductor coil 220 configured to receive power from a time-varying magnetic field generated by a transmitter inductor coil (not shown) of a wireless charging device (not shown). The power received by the receiver inductor coil 220 may be used to charge the electrical power source 212 wirelessly.

FIG. 2B illustrates an exploded view 225 of a controller 250 of one or more controllers 130 of the therapy kit 100 of FIG. 1A. The controller 250 includes an upper cap 252 that is configured to couple with a lower cap 272 to form a controller housing for the controller 250. The upper cap 252 may be fastened to the lower cap 272 using threaded fasteners 276. Furthermore, dust and water ingress protection to the controller 250 may be provided by a gasket 268. A pressure-sensitive switch 254 is configured to toggle the controller 250 between several operational states when pressure is applied to the respective user interface 131 of the controller 250. The controller 250 also includes a controller PCB 256.

Mounted on the controller PCB 256 are a processor 258, a memory unit 260 with machine-readable instructions, a communication interface 262, and a storage device 263. Furthermore, the controller 250 includes a rechargeable controller battery 266 configured to receive power from the base power terminals 133 through controller power terminals 274 and controller electrodes 264. The controller power terminals 274 may be embodied as holes capped with an electrically conducting material such as copper. The controller power terminals 274 therefore connect with the base power terminals 133 while charging the rechargeable controller battery 266, and with the patch power terminals 135 while activation of the one or more stimulation elements 129. Controller magnetic elements 270 are configured to magnetically couple with controller cavity magnetic elements 216 to enable magnetic coupling between the controller 250 and the corresponding controller cavity of the one or more controller cavities 132. The controller magnetic elements 270 and the controller cavity magnetic elements 216 may be selected from a group consisting of permanent magnets and electromagnets.

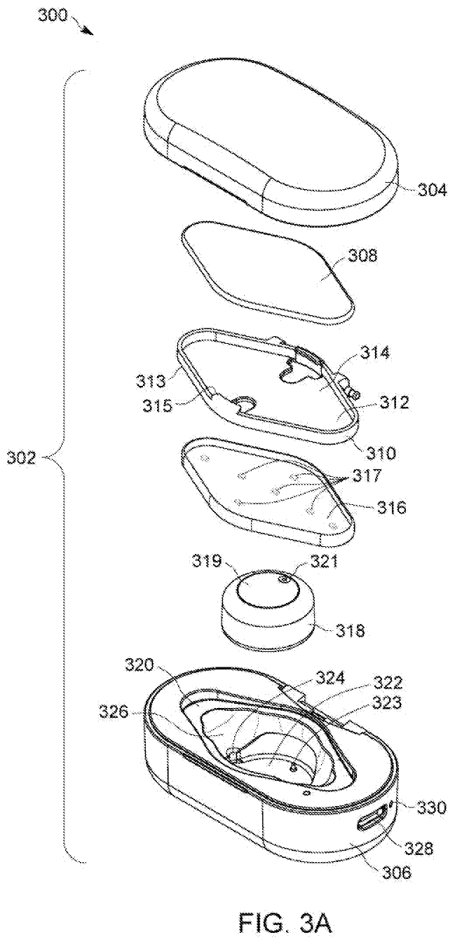

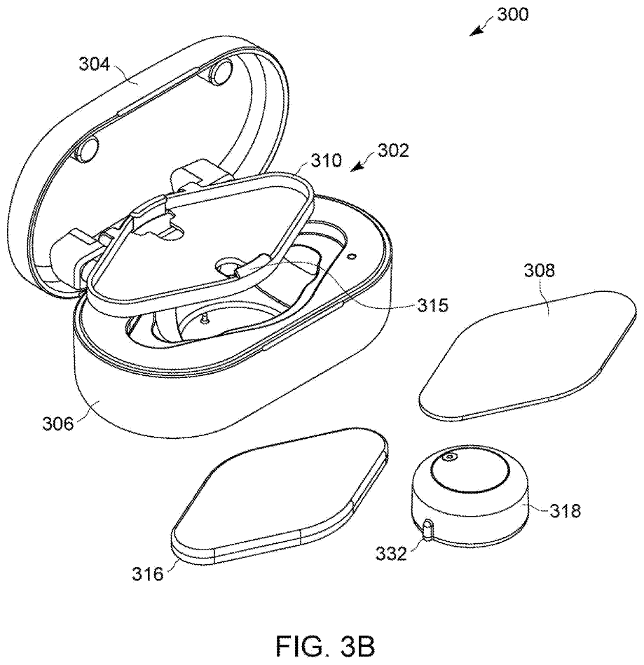

FIG. 3A illustrates an exploded view of a therapy kit 300 when viewed from the top, in accordance with another embodiment of the present invention. FIG. 3B illustrates a partially exploded view of the therapy kit 300 of FIG. 3A, when viewed from the front. Several constructional aspects of the therapy kit 300 may remain identical to those of the therapy kit 100, with an underlying difference that the therapy kit 100 includes two controllers 130, two stickers 122, two therapeutic patches 128, and therefore two controller cavities 132, two sticker cavities 126 and two patch cavities 134, respectively. Whereas the therapy kit 300 only includes one instance of the elements listed above. Other operational and material aspects of the therapy kit 300 may remain substantially identical to those of the therapy kit 100, and therefore the repetitive discussion of the other aspects has been omitted for brevity and clarity.

The therapy kit 300 includes a housing container 302. The housing container 302 includes a top lid 304 and a base portion 306. The top lid 304 is pivotably coupled to the base portion 306. The top lid 304 is configured to pivot between an open position and a closed position. Furthermore, an intermediate lid 310 is located between the top lid 304 and the base portion 306. The intermediate lid 310 is pivotably coupled to the top lid 304 and/or the base portion 306. The intermediate lid 310 is also configured to pivot between an open position and a closed position. Furthermore, the intermediate lid 310 includes an intermediate lid top portion 312 and an intermediate lid bottom portion 313. The intermediate lid top portion 312 includes a sticker cavity 314 configured to locate a sticker 308 therewithin. The intermediate lid top portion 312 further includes a lid coupling unit 315 configured to couple the intermediate lid 310 to the top lid 304 when the intermediate lid 310 is in an open position. The lid coupling unit 315 may enabled by magnets, adhesives, snap-fit arrangements, clips, or any other equivalent non-permanent coupling arrangements.

The base portion 306 includes a charging port 328 configured to receive electrical power from an external power source to charge an electrical power source (not shown) located within the base portion 306. The electrical power source of the base portion may include one or more rechargeable batteries and a first SoC indicator 330 may be configured to indicate the SoC of the one or more rechargeable batteries. The base portion 306 includes a controller cavity 322 with base power terminals 323 provided therewithin. The controller cavity 322 is configured to receive a controller 318 therewithin. The controller cavity 322 also includes an indentation as a mating feature 324 configured to mate with a protrusion serving as a complementary mating feature 332 of the controller 318 to prevent relative rotation between the controller cavity 322 and the controller 318. The controller cavity 322 has also been provided with a controller ejection enabling feature 326 in the form of extended tapered indentations on either side of the controller cavity 322. The controller 318 also includes a user interface 319 and a second SoC indicator 321 configured to indicate an SoC of one or more rechargeable controller batteries (not shown) of the controller 318. A patch cavity 320 is located above the controller cavity 322. The patch cavity is configured to receive a therapeutic patch 316. The therapeutic patch 316 further includes one or more stimulation elements 317.

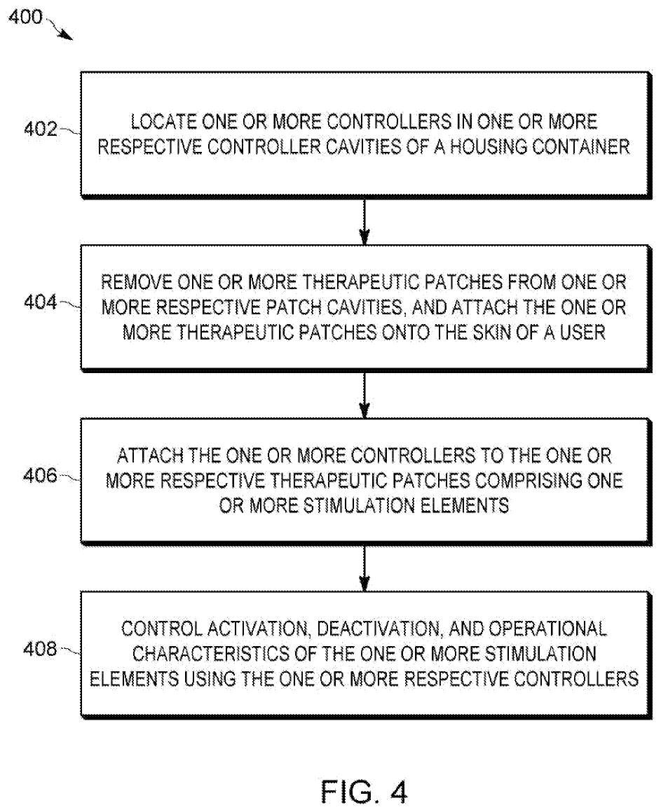

FIG. 4 illustrates a method 400 of utilizing the therapy kit 100 or 300, in accordance with an embodiment of the present invention. The method 400 begins at Step 402, when the one or more controllers 130 or 318 are located within the one or more respective controller cavities 132 or 322, respectively. The one or more controllers 130 or 318 may then receive electrical power through the base power terminals 133 or 323, respectively. at Step 404, the one or more therapeutic patches 128 or 316 are removed from the one or more respective patch cavities 134 or 320, respectively. The one or more therapeutic patches 128 or 316 are then attached to the skin of a user.

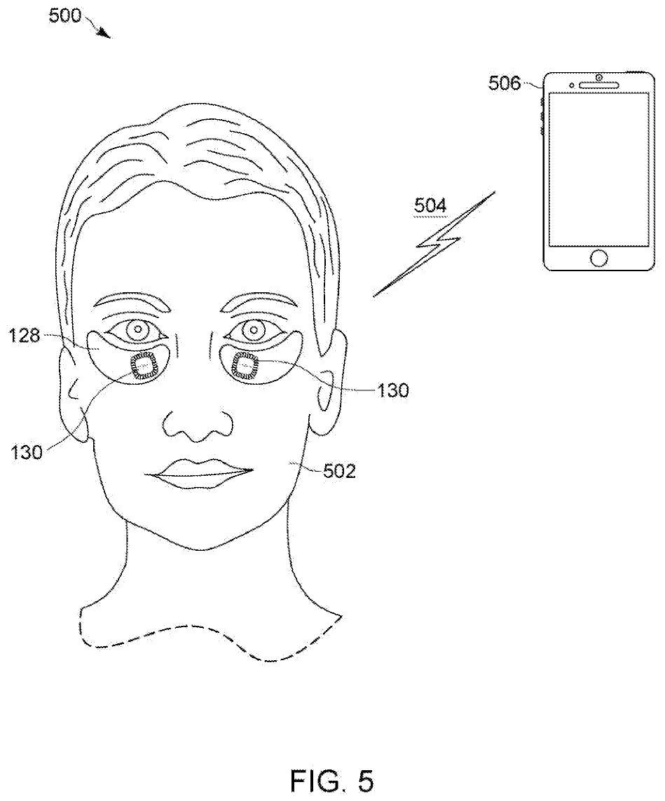

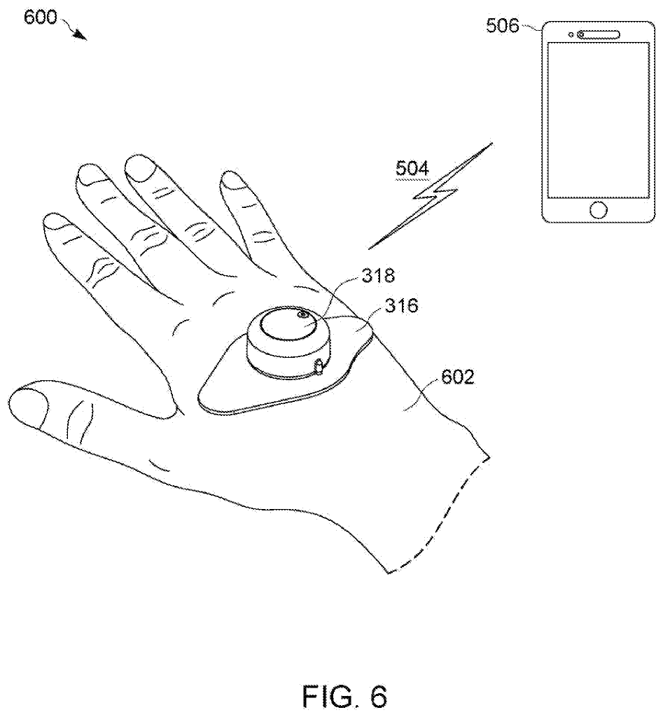

FIG. 5 illustrates an example application scenario 500 of the therapy kit 100 of FIGS. 1A-2B. The one or more therapeutic patches 128 are attached to a facial region 502 of the user. FIG. 6 illustrates an example application scenario 600 of the therapy kit 300 of FIGS. 3A and 3B. The therapeutic patch 316 is attached to an upper region of the hand 602 of a user. In several embodiments of the invention, each one of the one or more therapeutic patches 128 or 316 may include an adhesive agent on a surface configured to be attached to the skin of the user. In several alternate embodiments of the invention, the one or more therapeutic patches 128 or 316 may be attached through the one or more respective stickers 122 or 308, respectively. In that regard, each one of the one or more stickers 122 or 308 would have been provided with adhesive agents on respective opposite surfaces of the one or more stickers 122 or 308. Furthermore, in several embodiments, to enable attachment of the one or more therapeutic patches 128, the intermediate lid 124 may be pivoted from the closed position to the open position to reveal the mirror 144 fastened to the intermediate lid 124.

At Step 406, the one or more controllers 130 or 318 are attached to the one or more respective therapeutic patches 128 or 316, respectively. Furthermore, at Step 408, activation, deactivation, and operational characteristics of the one or more stimulation elements 129 or 317 may controlled using the one or more controller 130 or 318, respectively. Some of the non-limiting examples of the operational characteristics may include wavelength, intensity, period of pulses for the irradiation sources, temperature and time duration of activation of heating and cooling elements, frequency or acceleration of vibratory elements, frequency, and intensity of sound waves of the ultrasonic wave generators, applied voltage and current at the electrodes, etc. In that regard, in several embodiments of the present invention, the one or more controllers 130 or 318 may receive control input signals for modifying the operational characteristics through the one or more respective user interfaces 131 or 319, respectively. In several alternate embodiments of the invention, the one or more controllers 130 or 318 may receive the control input signals from a user computing device 504, through the respective communication interfaces 262 and a communication network 504. The user computing device 504 may be selected from a group consisting of smartphones, tablet PCs, desktop PCs, notebook PCs, and the like.

The embodiments of the invention as discussed above offer several advantages. For instance, all of the elements required for providing several different kinds of therapies, for example, therapeutic patches, stickers, controllers with user interfaces, and electrical power sources are contained within a single housing container. Furthermore, the sizes of the housing container and the entire therapy kit can be scaled up or down depending on the requirements. The therapeutic patches can be used to provide a very localized therapy to deal with localized skin infections or localized pain signals. Furthermore, the operational characteristics of stimulation elements can be controlled using user interfaces and smartphones connected through short-range networking such as Bluetooth without needing to connect to the Internet.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A therapy kit, comprising:
one or more therapeutic patches comprising one or more stimulation elements;
one or more controllers corresponding to the one or more respective therapeutic patches, the one or more controllers configured to control activation, deactivation, and operational characteristics of the one or more stimulation elements; and
a housing container comprising:
a base portion comprising an electrical power source, the base portion defining therewithin one or more controller cavities configured to receive the one or more respective controllers, and one or more patch cavities located above the respective one or more controller cavities, the one or more patch cavities configured to receive the one or more respective therapeutic patches, and
a top lid configured to be located above the base portion,
wherein the one or more controllers are configured to receive electrical power from the electrical power source through base power terminals provided within each one of the one or more respective controller cavities, and
wherein the one or more therapeutic patches are configured to receive electrical power from the one or more respective controllers, through patch power terminals provided with each one of the one or more respective therapeutic patches.

2. The therapy kit as claimed in claim 1, further comprising one or more stickers, each one of the one or more stickers provided with adhesive agents on two respective opposing surfaces for attaching the one or more respective therapeutic patches to skin of a user.

3. The therapy kit as claimed in claim 2, wherein the adhesive agents provided on the two respective opposing surfaces of at least one of the one or more stickers are diaphanous.

4. The therapy kit as claimed in claim 2, further comprising an intermediate lid configured to pivot between an open position and a closed position, such that, when in the closed position, the intermediate lid is located above the one or more patch cavities, wherein the intermediate lid comprises one or more sticker cavities configured to receive the one or more respective stickers.

5. The therapy kit as claimed in claim 4 wherein the intermediate lid comprises an intermediate lid top portion and an intermediate lid bottom portion, the intermediate lid top portion comprising the one or more sticker cavities.

6. The therapy kit as claimed in claim 5, wherein the intermediate lid bottom portion comprises a mirror fastened to the intermediate lid.

7. The therapy kit as claimed in claim 4, wherein the intermediate layer is pivotably coupled to the base portion and/or the top lid.

8. The therapy kit as claimed in claim 4, wherein the intermediate lid comprises one or more lid coupling units configured to couple the intermediate lid with the top lid when the intermediate lid is in the open position.

9. The therapy kit as claimed in claim 1, wherein each one of the one or more therapeutic patches comprises an adhesive agent on a surface configured to be attached to the skin of the user.

10. The therapy kit as claimed in claim 9, wherein the adhesive agent of the one or more therapeutic patches is diaphanous.

11. The therapy kit as claimed in claim 1, further comprising controller magnetic elements in each one of the one or more controllers and controller cavity magnetic elements under the one or more respective controller cavities, to enable magnetic couplings between the one or more controllers and the one or more respective controller cavities.

12. The therapy kit as claimed in claim 1, wherein each one of the one or more controllers comprises one or more respective rechargeable controller batteries and one or more respective second State of Charge (SoC) indicators, the one or more second SoC indicators configured to indicate SoC of the one or more respective rechargeable controller batteries.

13. The therapy kit as claimed in claim 1, wherein the electrical power source of the housing container comprises one or more rechargeable batteries, the housing container comprising a first State of Charge (SoC) indicator configured to indicate an SoC of the one or more rechargeable batteries.

14. The therapy kit as claimed in claim 1, wherein each one of the one or more controller cavities comprises a controller ejection enabling feature to enable removal of the one or more controllers from the one or more respective controller cavities.

15. The therapy kit as claimed in claim 1, wherein each one of the one or more controller cavities comprises a mating feature configured to mate with a complementary mating feature of a respective controller of the one or more controllers, to prevent relative rotation between the one or more controller cavities and the one or more respective controllers.

16. The therapy kit as claimed in claim 1, wherein the electrical power source of the base portion container comprises one or more rechargeable batteries, the base portion comprising a receiving inductor coil configured to receive power from a time-varying magnetic field generated by a transmitter inductor coil of a wireless charging device.

17. The therapy kit as claimed in claim 1, wherein the one or more stimulation elements are selected from a group consisting of irradiation sources, heating elements, cooling elements, vibration elements, ultrasonic wave generators, electrodes, and combinations thereof.

18. The therapy kit as claimed in claim 17, wherein the irradiation sources are configured to emit electromagnetic radiation in a wavelength range of 300 nm to 1200 nm.

19. The therapy kit as claimed in claim 1, wherein the top lid is pivotably coupled to the base portion.

20. The therapy kit as claimed in claim 1, wherein the housing container further comprises a charging port configured to receive electrical power from an external power source.

21. The therapy kit as claimed in claim 1, wherein the one or more controllers further comprise one or more respective communication interfaces configured to receive control input signals from a user computing device, through a communication network, to modify operational characteristics of the one or more stimulation elements.

22. The therapy kit as claimed in claim 1, wherein the one or more controllers further comprise one or more respective user interfaces configured to receive control input signals to modify operational characteristics of the one or more stimulation elements.

23. A therapy kit, comprising:
one or more therapeutic patches comprising one or more stimulation elements;
one or more stickers, each one of the one or more stickers provided with adhesive agents on two respective opposing surfaces for attaching the one or more respective therapeutic patches to skin of a user;
one or more controllers corresponding to the one or more respective therapeutic patches, the one or more controller configured to control activation, deactivation, and operational characteristics of the one or more stimulation elements; and
a housing container comprising:
a base portion comprising an electrical power source, the base portion defining therewithin:
one or more controller cavities configured to receive the one or more respective controllers, and
one or more patch cavities located above the respective one or more controller cavities, the one or more patch cavities configured to receive the one or more respective therapeutic patches,
an intermediate lid configured to pivot between an open position and a closed position, such that, when in the closed position, the intermediate lid is located above the one or more patch cavities, wherein the intermediate lid comprises one or more sticker cavities configured to receive the one or more respective stickers, and
a top lid configured to be located above the base portion and the intermediate lid,
wherein the one or more controllers are configured to receive electrical power from the electrical power source through base power terminals provided within each one of the one or more respective controller cavities,
wherein the one or more therapeutic patches are configured to receive electrical power from the one or more respective controllers, through patch power terminals provided with each one of the one or more respective therapeutic patches,
wherein the intermediate lid comprises an intermediate lid top portion and an intermediate lid bottom portion, the intermediate lid top portion comprising the one or more sticker cavities,
wherein the intermediate lid bottom portion comprises a mirror fastened to the intermediate lid, and
wherein the one or more controllers further comprise one or more respective user interfaces configured to receive control input signals to modify operational characteristics of the one or more stimulation elements.

24. The therapy kit as claimed in claim 23, wherein the one or more controllers further comprise one or more respective communication interfaces configured to receive control input signals from a user computing device, through a communication network, to modify operational characteristics of the one or more stimulation elements.

25. A method of utilizing a therapy kit, the method comprising:
locating one or more controllers in one or more respective controller cavities of a housing container of the therapy kit, such that, the one or more controllers receive electrical power from an electrical power source, through base power terminals provided within each one of the one or more respective controller cavities;
removing one or more therapeutic patches from one or more respective patch cavities from one or more respective patch cavities, and attaching the one or more therapeutic patches onto the skin of a user;
attaching the one or more controllers to the one or more respective therapeutic patches comprising one or more stimulation elements, such that, the one or more therapeutic patches receive electrical power through patch power terminals provided with each one of the one or more respective therapeutic patches; and
controlling activation, deactivation, and operational characteristics of the one or more stimulation elements using the one or more respective controllers.

26. The method as claimed in claim 25, wherein the one or more therapeutic patches are attached to the skin of the user through one or more respective stickers, each one of the one or more stickers provided with adhesive agents on respective opposite surfaces of the one or more stickers.

27. The method as claimed in claim 25, wherein each one of the one or more therapeutic patches comprises an adhesive agent on a surface configured to be attached to the skin of the user.

28. The method as claimed in claim 25, further comprising receiving, by the one or more controllers, control input signals through one or more respective user interfaces to modify operational characteristics of the one or more stimulation elements.

29. The method as claimed in claim 25, further comprising receiving, by the one or more controllers, control input signals from a user computing device, through one or more respective communication interfaces and a communication network, to modify operational characteristics of the one or more stimulation elements.

30. The method as claimed in claim 25, further comprising pivoting an intermediate lid of the housing container, from a closed position to an open position to reveal a mirror fastened to an intermediate lid bottom portion of the intermediate lid.
